(19) United States
(12) Patent Application Publication
(10) Pub. No.: US 2005/0272758 A1
(43) Pub. Date: Dec. 8, 2005

(54) Antineoplastic combinations of CCI-779 and rituximab

(75) Inventors: Eliel Bayever, Boston, MA (US); Laurence Moore, Newton, MA (US); Matthew L. Sherman, Newton, MA (US); Lee F. Allen, Chestnut Hill, MA (US); Lewis C. Strauss, Winnetka, IL (US)

Correspondence Address: HOWSON AND HOWSON;CATHY A. KODROFF, ONE SPRING HOUSE CORPORATE CENTER, BOX 457, SPRING HOUSE, PA, 19477, US

(73) Assignee: Wyeth, Madison, NJ (US)

(21) Appl. No.: 11/075,509

(22) Filed: Mar. 9, 2005

Related U.S. Application Data
(60) Provisional application No. 60/552,122, filed on Mar. 11, 2004.

(51) Int. Cl. A61K 31/4745

(52) U.S. Cl. 514/291

(57) ABSTRACT

This invention provides the use of a combination of CCI-779 and rituximab in the treatment of non-Hodgkin's lymphoma.

### CROSS-REFERENCE TO RELATED APPLICATION

This application claims the benefit under 35 USC 119(e) of U.S. Provisional Patent Application No. 60/552,122, filed Mar. 11, 2004.

### BACKGROUND OF THE INVENTION

This invention relates to the use of combinations of CCI-779 and rituximab for the treatment of non-Hodgkin's lymphoma.

CCI-779, is rapamycin 42-ester with 3-hydroxy-2-(hydroxymethyl)-2-methylpropionic acid, an ester of rapamycin which has demonstrated significant inhibitory effects on tumor growth in both in vitro and in vivo models. This compound is now known generically under the name temsirolimus. The preparation and use of hydroxyesters of rapamycin, including CCI-779, are described in U.S. Pat. Nos. 5,362,718 and 6,277,983.

CCI-779 exhibits cytostatic, as opposed to cytotoxic properties, and may delay the time to progression of tumors or time to tumor recurrence. CCI-779 is considered to have a mechanism of action that is similar to that of sirolimus. CCI-779 binds to and forms a complex with the cytoplasmic protein FKBP, which inhibits an enzyme, mTOR (mammalian target of rapamycin, also known as FKBP12-rapamycin associated protein [FRAP]). Inhibition of mTOR's kinase activity inhibits a variety of signal transduction pathways, including cytokine-stimulated cell proliferation, translation of mRNAs for several key proteins that regulate the G1 phase of the cell cycle, and IL-2-induced transcription, leading to inhibition of progression of the cell cycle from G1 to S. The mechanism of action of CCI-779 that results in the G1 -S phase block is novel for an anticancer drug. CCI-779 has been describes as a sole agent in connection with the treatment of mantle cell lymphoma.

Rituximab, an anti-CD20 monoclonal antibody, is approved for treatment of patients with relapsed or refractory, low-grade or follicular, CD20-positive, B-cell non-Hodgkin's lymphoma in the United States. In Europe, it is also approved for this indication, as well as for use in combination with CHOP (cyclophosphamide, doxorubicin, vincristine, prednisone) for the most common aggressive non-Hodgkin's lymphoma, diffuse large cell. However, rituximab is associated with serious side effects including acute renal failure, severe mucocutaneous reactions, and cardiovascular distress.

What is needed is an improved therapy for CD20+ and mantle cell lymphoma and for other non-Hodgkin's lymphoma.

### DETAILED DESCRIPTION OF THE INVENTION

This invention provides the use of combinations of CCI-779 and rituximab in the treatment of non-Hodgkin's lymphoma.

This invention also provides use of combinations of other mTOR inhibitors such as rapamycin and 42-O-(2-hydroxy)ethyl rapamycin and rituximab in the treatment of non-Hodgkin's lymphoma. The preparation of 42-O-(2-hydroxy)ethyl rapamycin is described in U.S. Pat. No. 5,665,772, which is hereby incorporated by reference.

As used in accordance with this invention, the term “treatment” means treating a mammal having a non-Hodgkin's lymphoma by providing said mammal an effective amount of a combination of CCI-779 and rituximab with the purpose of inhibiting growth of the non-Hodgkin's lymphoma in such mammal, eradication of the non-Hodgkin's lymphoma, or palliation of the mammal.

Non-Hodgkin's lymphomas are cancers of lymphoid tissue (lymph nodes, spleen, and other organs of the immune system. Non-Hodgkin's lymphoma includes, slow-growing lymphomas and lymphoid leukemias of B-cell or T-cell subtypes, such as the B-cell lymophomas, such as B-cell chronic lymphocytic leukemia (B-CLL)/small lymphocytic lymphoma (SLL), lymphoplasmacytoid lymphoma, follicle center lymphoma, follicular small cleaved cell (FSC), follicular mixed cell (FM), marginal zone B-cell lymphoma, hairy cell leukemia, plasmacytoma/myeloma and T-cell lymphomas, including, large granular lymphocyte leukemia, adult T-Cell leukemia/lymphoma (ATL/L), mycosis fungoides/sezary syndrome. Also included are moderately aggressive lymphomas and lymphoid leukemias of B-cell original, e.g., B-cell prolymphocytic leukemia (B-PLL), mantle cell lymphoma, follicle center lymphoma, follicular small cleaved cell (FSC), follicle center lymphoma (follicular large cell) or T-cell origin, T-cell chronic lymphocytic leukemia/prolymphocytic leukemia (T-CLL/PLL), adult T-Cell leukemia/lymphoma (ATL/L) [chronic], angiocentric lymphoma, angioimmunoblastic lymphoma, aggressive lymphomas including, B-cell large B-cell lymphoma, peripheral T-cell lymphomas, intestinal T-cell lymphoma, anaplastic large cell lymphoma, highly aggressive lymphomas and lymphoid leukemias, including precursor B-lymphoblastic leukemia/lymphoma (PB-LBL/L), Burkitt's lymphoma, high-grade B-Cell lymphoma, Burkitt's-like, and precursor T-lymphoblastic leukemia/lymphoma (T-LBL/L), adult T-cell leukemia/lymphoma (ATLL) [acute and lymphomatous], slow-growing (Low Grade) Lymphomas of the B-cell types, e.g., small lymphocytic/pro-lymphocytic lymphoma (SLL), follicular lymphoma (few large cells), lymphoplasmacytoid lymphoma, marginal zone lymphoma, and slow-growing lymphomas of the T-cell subtypes, for example, large granular lymphocyte leukemia, adult T-cell leukemia/lymphoma (ATL/L ), and mycosis fungoides/Sezary syndrome.

As used in accordance with this invention, the term “providing,” with respect to providing CCI-779 and rituximab, means either directly administering CCI-779, or administering a prodrug, derivative, or analog which will form an effective amount of CCI-779 within the body, along with rituximab directly, or administering a prodrug, derivative, or analog which will form an effective amount of rituximab in the body. Use of a combination of CCI-779 and rituximab also provides for the use of combinations of each of the agents in which one or both of the agents is used at subtherapeutically effective dosages.

Subtherapeutically effective dosages may be readily determined by one of skill in the art, in view of the teachings herein. In one embodiment, the subtherapeutically effective dosage is a dosage which is effective at a lower dosage when used in the combination regimen of the invention, as compared to the dosage that is effective when used alone.

The preparation of CCI-779 is described in U.S. Pat. No. 5,362,718, which is hereby incorporated by reference. A regiospecific synthesis of CCI-779 is described in U.S. Pat. No. 6,277,983, which is hereby incorporated by reference. Still another regiospecific method for synthesis of CCI-779 is described in U.S. patent application Ser. No. 10/903,062, filed Jul. 30, 2004, and its counterpart, International Patent Application PCT/US2004/22860, filed Jul. 15, 2004. Rituximab is commercially available as Rituxan® rituximab.

The combinations of the invention may be in the form of a kit of parts. The invention therefore includes a product containing an mTOR inhibitor and rituximab as a combined preparation for simultaneous, separate or sequential use in treating non-Hodgkins lymphoma in a mammal in need thereof. In one embodiment, a product contains CCI-779 and rituximab as a combined preparation for simultaneous, separate or sequential use in treating non-Hodgkins lymphoma in a mammal in need thereof.

The invention also includes a pharmaceutical pack containing a course of treatment of non-Hodgkins lymphoma for one individual mammal, wherein the pack contains units of an mTOR inhibitor in unit dosage form and units of rituximab in unit dosage form. In one embodiment, a pharmaceutical pack contains a course of treatment of non-Hodgkins lymphoma for one individual mammal, wherein the pack contains units of CCI-779 in unit dosage form and units of rituximab in unit dosage form.

While the components of the invention may be delivered via the same route, a product or pack according to the invention may contain an mTOR inhibitor, such as CCI-779, for delivery by a different route than that of the rituximab, e.g., one component may be delivered orally, while the other is administered intravenously. In one embodiment, CCI-779 is prepared for oral delivery and rituximab is prepared for intravenous delivery. Other variations would be apparent to one skilled in the art and are contemplated within the scope of the invention.

As is typical with chemotherapy, dosage regimens are closely monitored by the treating physician, based on numerous factors including the severity of the disease, response to the disease, any treatment related toxicities, age, and health of the patient. Based on the results obtained with CCI-779, it is projected that initial i.v. infusion dosages will be between about 25 and 175 mg when administered on a weekly dosage regimen. Other dosage regimens and variations are foreseeable, and will be determined through physician guidance. It is preferred that CCI-779 is administered by i.v. infusion or orally, preferably in the form of tablets or capsules. Other routes of administration are also feasible, such as via implants, parenterally (besides i.v., such as intraperitoneal and subcutaneous injections), rectally, intranasally, vaginally, and transdermally.

For rituximab, single doses and multiple doses are contemplated. In one embodiment, single doses are provided intravenously at concentrations of from 10 to 500 mg/m², from 50 to 500 mg/m², from 100 to 500 mg/m², or from 250 to 500 mg/m². In another embodiment, it is projected that initial dosages will be from about 350 to about 400 mg/m²/week intravenously for from 4-8 weeks, or from 4, 6, or 8 weeks, or 375 mg/m²/week intravenously for from 4-8 weeks, or from 4, 6, or 8 weeks, with potential readministration every 3 to 6 months. Other dosage regimens and variations are foreseeable, and will be determined through physician guidance. It is preferred that rituximab is administered subcutaneously.

As described herein, subtherapeutically effective amounts of rituximab and CCI-779 may be used to achieve a therapeutic effect when administered in combination. For example, rituximab may be provided at dosages of 5 to 50% lower, 10 to 25% lower, or 15 to 20% lower, when provided along with CCI-779. For example, a resulting rituximab dosage can be from about 315 to 380 mg/m²/week intravenously, or about 350 mg/m²/week, or lower. Use of subtherapeutically effective amounts of rituximab is expected to reduce the side-effects of rituximab treatment.

Dosage regimens are expected to vary according to the route of administration. For example, dosages for oral administration are often up to five to tenfold greater than for i.v. administration, i.e., 125 mg to 1000 mg/week for CCI-779. It is anticipated that the CCI-779 plus rituximab combination may be administered as the sole active chemotherapeutic agents, or may be part of a chemotherapeutic regimen containing other antineoplastic agents. The use of concomitant chemotherapeutic agents often allows for dosage reduction of each particular agent, thereby increasing the safety margin of the particular agents. As the combinations of this invention contain at least two active antineoplastic agents, the use of such combinations also provides for the use of combinations of each of the agents in which one or both of the agents is used at subtherapeutically effective dosages. For example, CCI-779 may be administered at a dosage 5 to 50% lower, 10 to 25% lower, or 15 to 20% lower, than when delivered as a sole agent.

As used in this invention, the combination regimen can be given simultaneously or can be given in a staggered regimen, with CCI-779 being given at a different time during the course of chemotherapy than the rituximab. This time differential may range from several minutes, hours, days, weeks, or longer between administration of the two agents. Therefore, the term combination (or combined) does not necessarily mean administered at the same time or as a unitary dose, but that each of the components are administered during a desired treatment period. The agents may also be administered by different routes.

Oral formulations containing the active compounds of this invention may comprise any conventionally used oral forms, including tablets, capsules, buccal forms, troches, lozenges and oral liquids, suspensions or solutions. Capsules may contain mixtures of the active compound(s) with inert fillers and/or diluents such as the pharmaceutically acceptable starches (e.g. corn, potato or tapioca starch), sugars, artificial sweetening agents, powdered celluloses, such as crystalline and microcrystalline celluloses, flours, gelatins, gums, etc. Useful tablet formulations may be made by conventional compression, wet granulation or dry granulation methods and utilize pharmaceutically acceptable diluents, binding agents, lubricants, disintegrants, surface modifying agents (including surfactants), suspending or stabilizing agents, including, but not limited to, magnesium stearate, stearic acid, talc, sodium lauryl sulfate, microcrystalline cellulose, carboxymethylcellulose calcium, polyvinylpyrrolidone, gelatin, alginic acid, acacia gum, xanthan gum, sodium citrate, complex silicates, calcium carbonate, glycine, dextrin, sucrose, sorbitol, dicalcium phosphate, calcium sulfate, lactose, kaolin, mannitol, sodium chloride, talc, dry starches and powdered sugar. Preferred surface modifying agents include nonionic and anionic surface modifying agents. Representative examples of surface modifying agents include, but are not limited to, poloxamer 188, benzalkonium chloride, calcium stearate, cetostearyl alcohol, cetomacrogol emulsifying wax, sorbitan esters, colloidal silicon dioxide, phosphates, sodium dodecylsulfate, magnesium aluminum silicate, and triethanolamine. Oral formulations herein may utilize standard delay or time release formulations to alter the absorption of the active compound(s). The oral formulation may also consist of administering the active ingredient in water or a fruit juice, containing appropriate solubilizers or emulsifiers as needed. Preferred oral formulations for rapamycin 42-ester with 3-hydroxy-2-(hydroxymethyl)-2-methylpropionic acid are described in US patent Publication No. 2004/0077677 A1, published Apr. 22, 2004, which is hereby incorporated by reference.

In some cases it may be desirable to administer the compounds directly to the airways in the form of an aerosol.

The compounds may also be administered parenterally or intraperitoneally. Solutions or suspensions of these active compounds as a free base or pharmacologically acceptable salt can be prepared in water suitably mixed with a surfactant such as hydroxy-propylcellulose. Dispersions can also be prepared in glycerol, liquid polyethylene glycols and mixtures thereof in oils. Under ordinary conditions of storage and use, these preparations contain a preservative to prevent the growth of microorganisms.

The pharmaceutical forms suitable for injectable use include sterile aqueous solutions or dispersions and sterile powders for the extemporaneous preparation of sterile injectable solutions or dispersions. In all cases, the form must be sterile and must be fluid to the extent that easy syringability exists. It must be stable under the conditions of manufacture and storage and must be preserved against the contaminating action of microorganisms such as bacteria and fungi. The carrier can be a solvent or dispersion medium containing, for example, water, ethanol, polyol (e.g.,. glycerol, propylene glycol and liquid polyethylene glycol), suitable mixtures thereof, and vegetable oils. Preferred injectable formulations for rapamycin 42-ester with 3-hydroxy-2-(hydroxymethyl)-2-methylpropionic acid are described in US patent Publication No. 2004/0167152 A1, published Aug. 26, 2004, which is hereby incorporated by reference.

For the purposes of this disclosure, transdermal administrations are understood to include all administrations across the surface of the body and the inner linings of bodily passages including epithelial and mucosal tissues. Such administrations may be carried out using the present compounds, or pharmaceutically acceptable salts thereof, in lotions, creams, foams, patches, suspensions, solutions, and suppositories (rectal and vaginal).

Transdermal administration may be accomplished through the use of a transdermal patch containing the active compound and a carrier that is inert to the active compound, is non toxic to the skin, and allows delivery of the agent for systemic absorption into the blood stream via the skin. The carrier may take any number of forms such as creams and ointments, pastes, gels, and occlusive devices. The creams and ointments may be viscous liquid or semisolid emulsions of either the oil-in-water or water-in-oil type. Pastes comprised of absorptive powders dispersed in petroleum or hydrophilic petroleum containing the active ingredient may also be suitable. A variety of occlusive devices may be used to release the active ingredient into the blood stream such as a semi-permeable membrane covering a reservoir containing the active ingredient with or without a carrier, or a matrix containing the active ingredient. Other occlusive devices are known in the literature.

Suppository formulations may be made from traditional materials, including cocoa butter, with or without the addition of waxes to alter the suppository's melting point, and glycerin. Water soluble suppository bases, such as polyethylene glycols of various molecular weights, may also be used.

All patents, patent publications, articles, and other documents referenced herein are incorporated by reference. It will be clear to one of skill in the art that modifications can be made to the specific embodiments described herein without departing from the scope of the invention.

1. A method of treating non-Hodgkins lymphoma in a mammal in need thereof, which comprises providing to said mammal an effective amount of a combination comprising CCI-779 and rituximab.

2. The method according to claim 1, wherein either CCI-779 or rituximab, or both are provided in subtherapeutically effective amounts.

3. A method of treating non-Hodgkins lymphoma in a mammal in need thereof, which comprises providing to said mammal an effective amount of a combination comprising an mTOR inhibitor and rituximab.

4. The method according to claim 1, wherein either the mTOR inhibitor, rituximab, or both are provided in subtherapeutically effective amounts.

5. The method according to claim 3 or claim 4, wherein the mTOR inhibitor is rapamycin.

6. The method according to claim 3 or claim 4, wherein the mTOR inhibitor is 42-O-(2-hydroxy)ethyl rapamycin.

7. A product containing CCI-779 and rituximab as a combined preparation for simultaneous, separate or sequential use in treating non-Hodgkins lymphoma in a mammal.

8. A product containing an mTOR inhibitor and rituximab as a combined preparation for simultaneous, separate or sequential use in treating non-Hodgkins lymphoma in a mammal.

9. A pharmaceutical pack containing a course of treatment of non-Hodgkins lymphoma for one individual mammal, wherein the pack contains (a) at least one unit of CCI-779 and (b) at least one unit of rituximab in unit dosage form.

10. A pharmaceutical pack containing a course of treatment of non-Hodgkins lymphoma for one individual mammal, wherein the pack contains (a) at least one unit of an mTOR inhibitor and (b) at least one unit of rituximab in unit dosage form.

11. A pharmaceutical composition useful in treating non-Hodgkins lymphoma in a mammal, the composition comprising (a) at least one unit of CCI-779 and (b) at least one unit of rituximab in unit dosage form, and a pharmaceutically acceptable carrier.

12. A pharmaceutical composition useful in treating non-Hodgkins lymphoma in a mammal, the composition comprising (a) at least one unit of an mTOR inhibitor and (b) at least one unit of rituximab in unit dosage form, and a pharmaceutically acceptable carrier.
